(19) United States
(12) Patent Application Publication
(10) Pub. No.: US 2013/0195309 A1
(43) Pub. Date: Aug. 1, 2013

(54) EAR WARMER

(71) Applicants: Amy TANKERSLEY, Baltimore, MD (US); Paul REEDER, Worthington, OH (US); Erika BRAUN, Columbus, OH (US); George CAMPBELL, San Pedro, CA (US); Jennifer GOFF, Williamsport, PA (US)

(72) Inventors: Amy TANKERSLEY, Baltimore, MD (US); Paul REEDER, Worthington, OH (US); Erika BRAUN, Columbus, OH (US); George CAMPBELL, San Pedro, CA (US); Jennifer GOFF, Williamsport, PA (US)

(73) Assignee: 180S, INC., Baltimore, MD (US)

(21) Appl. No.: 13/565,470

(22) Filed: Aug. 2, 2012

Related U.S. Application Data
(60) Provisional application No. 61/514,470, filed on Aug. 2, 2011.

(51) Int. Cl. A61F 11/14; H04R 1/10

(52) U.S. Cl. CPC A61F 11/14; H04R 1/1075
USPC 381/378; 2/209

(57) ABSTRACT

An adjustable ear warmer is disclosed. The ear warmer has a band and ear portions coupled to the band. The band and the ear portions are slideably coupled so that the band and the ear portions can move relative to each other.

### CROSS REFERENCE TO OTHER APPLICATIONS

This application claims priority benefit of U.S. provisional application no. 61/514,470, filed Aug. 2, 2011, which is incorporated by reference herein in its entirety.

### BACKGROUND OF THE INVENTION

This invention relates generally to an ear warmer. More specifically, the present invention relates to an adjustable ear warmer and a method of making an adjustable ear warmer.

### SUMMARY OF THE INVENTION

In one embodiment, an ear warmer includes a frame that is configured to be worn by a user. The frame is configured to extend around the back of the head and/or neck of the user. The ear warmer includes a fabric portion that is coupled to the frame and engageable with an ear of the user when the user wears the ear warmer. The ear warmer also includes another fabric portion that is coupled to the frame and engageable with an ear of the user when the user wears the ear warmer.

In one embodiment, the frame of the ear warmer includes two ear portions located at opposite ends of the frame. The frame includes a band portion that is located between the ear portions and coupled thereto. The overall length of the frame can vary by movement of the ear portions relative to the band portion.

In one embodiment, the ear warmer includes an adjustment mechanism or coupler to which the ear portions and any band portions or ends connected to the ear portions are coupled. The ear portions and/or band ends are slideably coupled to the adjustment mechanism or coupler and are independently movable relative thereto. In one example, the coupler is slideably received into openings formed in the ear portions and/or band ends.

In one embodiment, an ear warmer comprises a first ear portion disposable proximate to a first ear of a user, a first band portion coupled to the first ear portion, the first band portion having an end distal to the first ear portion; a second ear portion disposable proximate to a second ear of the user; a second band portion coupled to the second ear portion, the second band portion having an end distal to the second ear portion; and an adjustment mechanism that permits adjustment to the overall length of the ear warmer, the end of the first band portion being slideably coupled to the adjustment mechanism, and the end of the second band portion being slideably coupled to the adjustment mechanism.

In one implementation, the first ear portion and the first band portion are integrally formed together.

In another implementation, the end of the first band portion includes an opening into which a first portion of the adjustment mechanism is inserted, and the end of the second band portion includes an opening into which a second portion of the adjustment mechanism is inserted.

In another implementation, the first ear portion is pivotally coupled to the first band portion, and the second ear portion is pivotally coupled to the second band portion.

In another implementation, the first ear portion includes a wireless communication device coupled thereto. The wireless communication device is Bluetooth enabled.

In one embodiment, an ear warmer comprises a band being disposable around a portion of a user's head, the band having a first end and a second end opposite the first end, the band including a first adjustment member extending from the first end and a second adjustment member extending from the second end, a first ear portion slideably mounted on the first adjustment member, the first ear portion being placeable in a deployed position and in a collapsed position relative to the band, and a second ear portion slideably mounted on the second adjust member, the second ear portion being placeable in a deployed position and in a collapsed position relative to the band, wherein the overall length of the ear warmer changes as at least one of the first ear portion or the second ear portion moves relative to the band.

In one implementation, the first ear portion can be pivoted inwardly from its deployed position to its collapsed position, and the second ear portion can be pivoted inwardly from its deployed position to its collapsed position.

In an alternative implementation, the first ear portion includes a first fabric portion removably coupled thereto, and the second ear portion includes a second fabric portion removably coupled thereto.

In another embodiment, an ear warmer comprises a band configured to be disposed around a portion of a user's head, a first ear portion coupled to the band, the first ear portion including a wall defining an opening, the wall including a groove formed therein, a first fabric portion removably coupleable to the first ear portion, the first fabric portion including a mounting component and a fabric member coupled to the mounting component, the mounting component being engageable with the groove to couple the first fabric portion to the first ear portion, and a second ear portion coupled to the band.

In one implementation, the first ear portion has an outer surface and an inner surface, the wall extends between the outer surface and the inner surface, and the groove is formed in the wall and spaced from the outer surface and the inner surface.

In another implementation, the second ear portion includes a wall defining an opening, the wall of the second ear portion includes a groove formed therein, and the ear warmer further comprises: a second fabric portion removably coupleable to the second ear portion, the second fabric portion including its own mounting component and fabric member mounted to its mounting component, the mounting component of the second fabric portion being engageable with the groove of the second ear potion to couple the second fabric portion to the second ear portion.

### BRIEF DESCRIPTION OF THE DRAWINGS

FIG. 1 is a side view of a user wearing an ear warmer according to the present invention.

FIG. 2 is a side view of an embodiment of an ear warmer according to the present invention.

FIG. 3 is a rear perspective view of the ear warmer illustrated in FIG. 2.

FIG. 4 is a side view of another embodiment of an ear warmer according to the present invention.

FIG. 5 is a rear perspective view of another embodiment of an ear warmer according to the present invention.

FIG. 6 is a top view of the ear warmer illustrated in FIG. 5.

FIG. 7 is an exploded rear perspective view of the ear warmer illustrated in FIG. 5.

FIG. 8 is an exploded front perspective view of the ear warmer illustrated in FIG. 5.

FIG. 9 is an exploded top view of the ear warmer illustrated in FIG. 5.

FIG. 10 is a side view of another embodiment of an ear warmer according to the present invention.

FIG. 11 is a perspective view of the ear warmer illustrated in FIG. 10.

FIG. 12 is a top view of another embodiment of an ear warmer according to the present invention.

FIG. 13 is an exploded top view of the ear warmer illustrated in FIG. 12.

FIG. 14 is an exploded rear perspective view of the ear warmer illustrated in FIG. 12.

FIG. 15 is an exploded front perspective view of the ear warmer illustrated in FIG. 12.

FIG. 16 is a side view of another embodiment of an ear warmer according to the present invention.

FIG. 17 is a rear perspective view of the ear warmer illustrated in FIG. 16.

FIG. 18 is an exploded rear perspective view of another embodiment of an ear warmer according to the present invention.

FIG. 19 is an exploded front perspective view of the ear warmer illustrated in FIG. 18.

FIG. 20 is an exploded top view of the ear warmer illustrated in FIG. 18.

Like reference numerals have been used to identify like elements throughout this disclosure.

### DETAILED DESCRIPTION

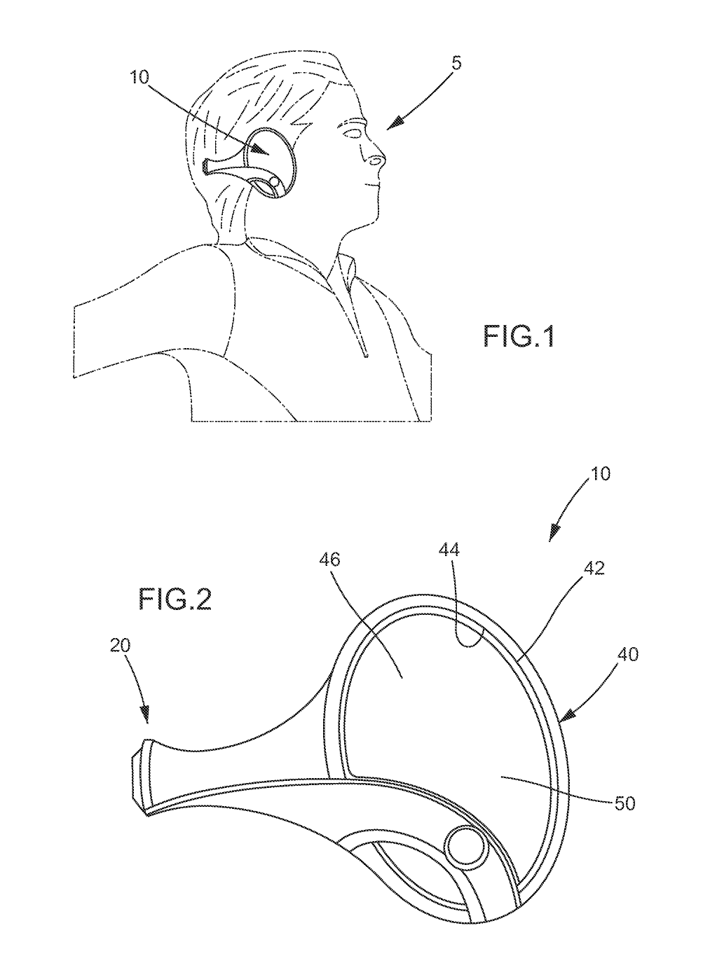

An ear warmer according to the present invention can be worn on the head of a user as illustrated in FIG. 1. The ear warmer is configured to cover the wearer's ears to provide comfort as well as warmth and protection from the elements. As shown in FIG. 1, the ear warmer 10 extends around the back of the user's head 5. The ear warmer 10 includes ear portions that are located so as to cover the ears of the user or wearer of the ear warmer 10. The ear portions can be referred to alternatively as ear frame members or ear members.

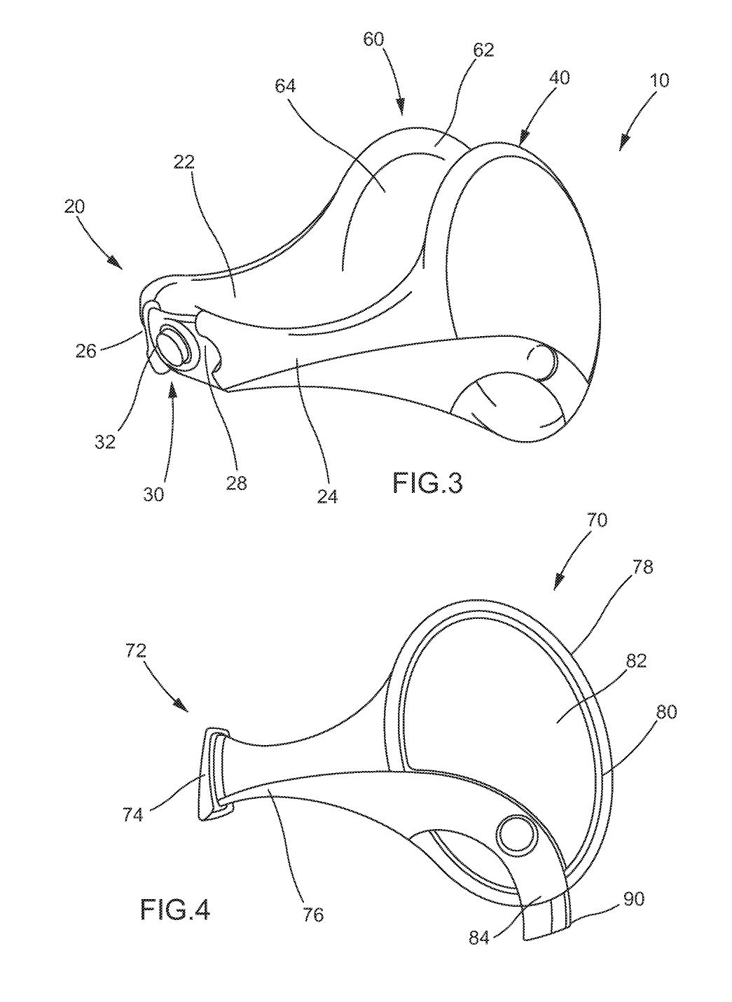

An embodiment of an ear warmer is illustrated in FIGS. 2 and 3. In this embodiment, the ear warmer 10 includes a band portion 20 that extends around the back of the user's head. The ear warmer 10 includes ear portions 40 and 60 located at opposite ends of the band portion 20. The band portion 20 is formed of the band ends 22 and 24 that are coupled to an adjustment mechanism 30 as shown in FIG. 3. The band ends 22 and 24 have slots formed therein that receive portions of the adjustment mechanism 30, which allows the band ends 22 and 24 to slide on and relative to the adjustment mechanism 30.

In one embodiment, the adjustment mechanism 30 includes an actuator 32 that can be manipulated to release a mechanism that retains the band ends 22 and 24 in their positions relative to each other. For example, the actuator 32 can be a button that is pressed to release the band ends 22 and 24 so that the band ends 22 and 24 can be moved. The actuator 32 permits one-handed adjustment of the ear warmer 10 while the ear warmer 10 is being worn by the user. In one implementation, the band ends 22 and 24 have notches 26 and 28, respectively, that allow for the band ends 22 and 24 to slide proximate to the actuator 32.

As shown in FIG. 2, ear portion 40 includes a frame 42 that has an internal edge 44 that defines an opening 46 that extends through the frame 42. The opening 46 is covered by fabric portion 50. The fabric portion 50 can be formed of one or more layers of material that are coupleable to the ear portion 40. In one embodiment, the fabric portion 50 can be releaseably coupled to the frame 42 of the ear portion 40. In another embodiment, the fabric portion 50 can be fixedly coupled to the frame 42 of the ear portion 40. Similarly, ear portion 60 includes a frame 62 to which another fabric portion 64 is coupled. The fabric portions 50 and 64 can be a high visibility fabric with reflective properties.

The band ends 22 and 24 and the ear portions 40 and 60 are molded articles made of a plastic material. Band end 22 and ear portion 40 are integrally formed and have substantially continuous surfaces on their outer sides, their inner sides, their upper sides, and their lower sides. Similarly, band end 24 and ear portion 60 are integrally formed and have substantially continuous surfaces on their outer sides, their inner sides, their upper sides, and their lower sides.

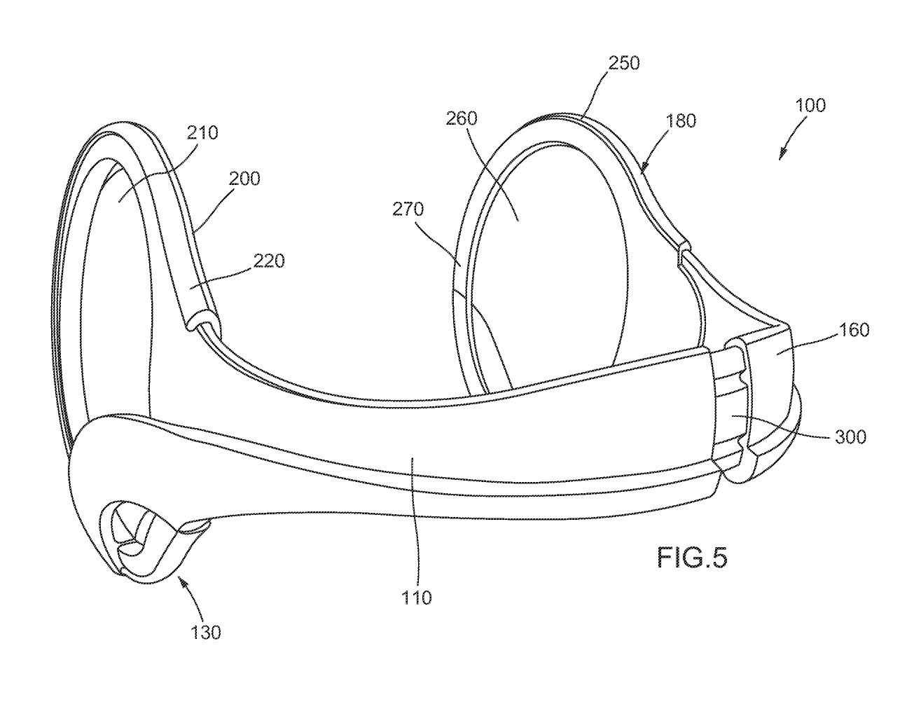

Referring to FIG. 4, another embodiment of an ear warmer according to the present invention is illustrated. In this embodiment, the ear warmer 70 includes a band portion 72 that has an adjustment mechanism 74. The band portion 72 is formed in part by frame portion 76 which includes an ear portion 78 and in part by another frame portion (not shown in FIG. 4) that is a reverse image of frame portion 76 and that includes an ear portion. The ear portion 78 is defined by frame 80 to which a fabric portion 82 is coupled.

In this embodiment, the ear warmer 70 includes an electronic component or wireless communication device 90 coupled thereto. The electronic component 90 is a BLUETOOTH enabled component that can be used with an external electronic device, such as a mobile digital device. The ear warmer 70 can include speakers proximate to each of the ear portions that can generate audible outputs for the wearer of the ear warmer 70. The speakers can be connected to the electronic component 90 in a wired manner or a wireless manner.

The electronic component 90 can be coupled to a lower portion 84 of the frame portion 76. For example, the lower portion 84 can include a notch into which the electronic component 90 can be snapped or coupled.

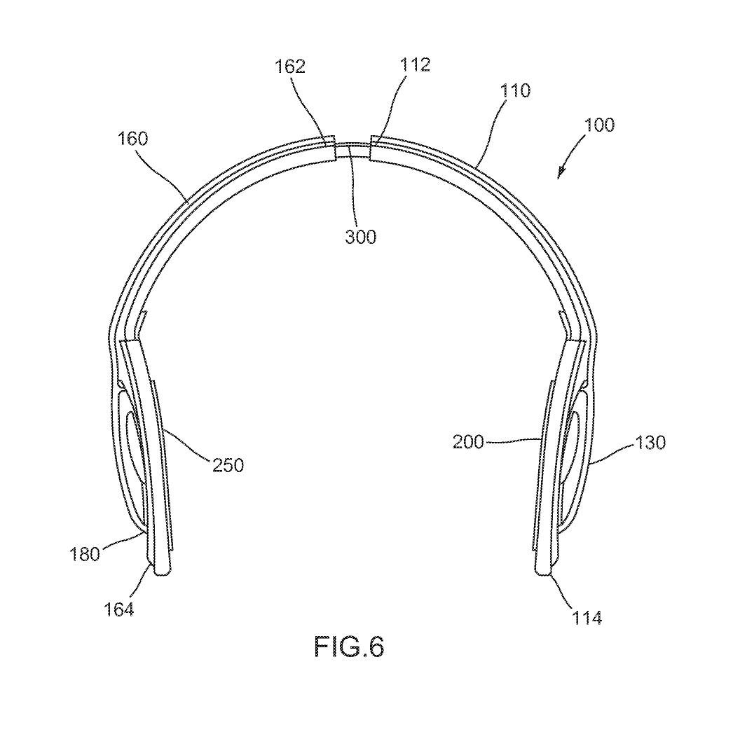

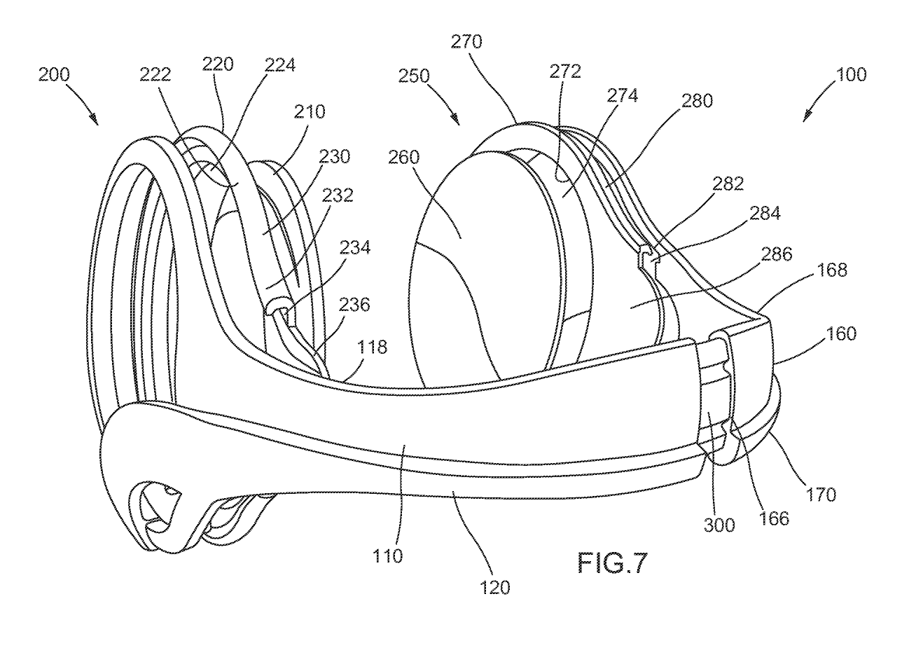

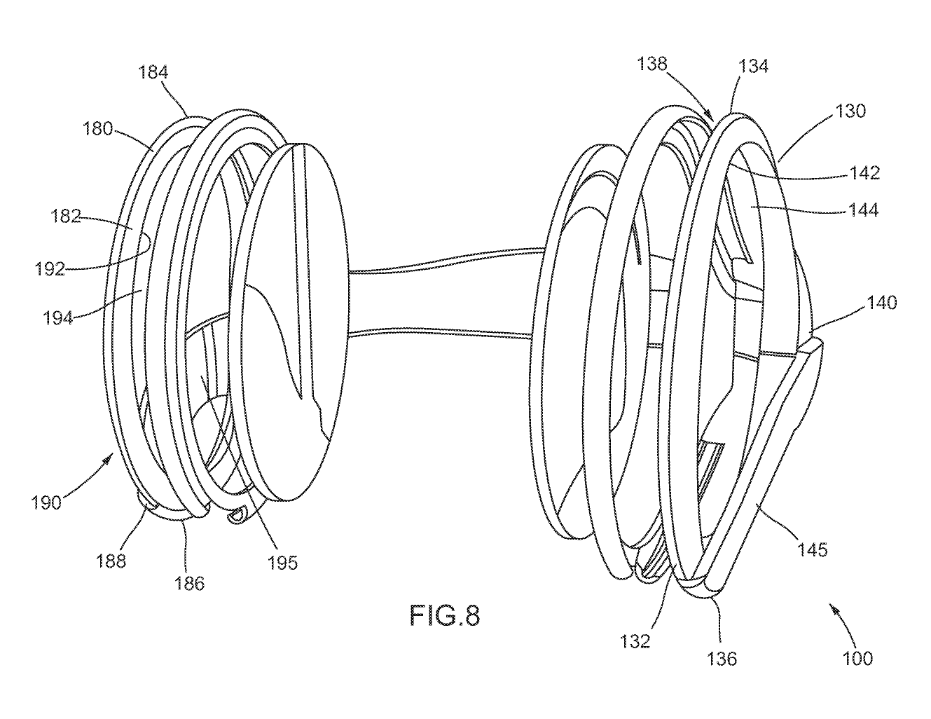

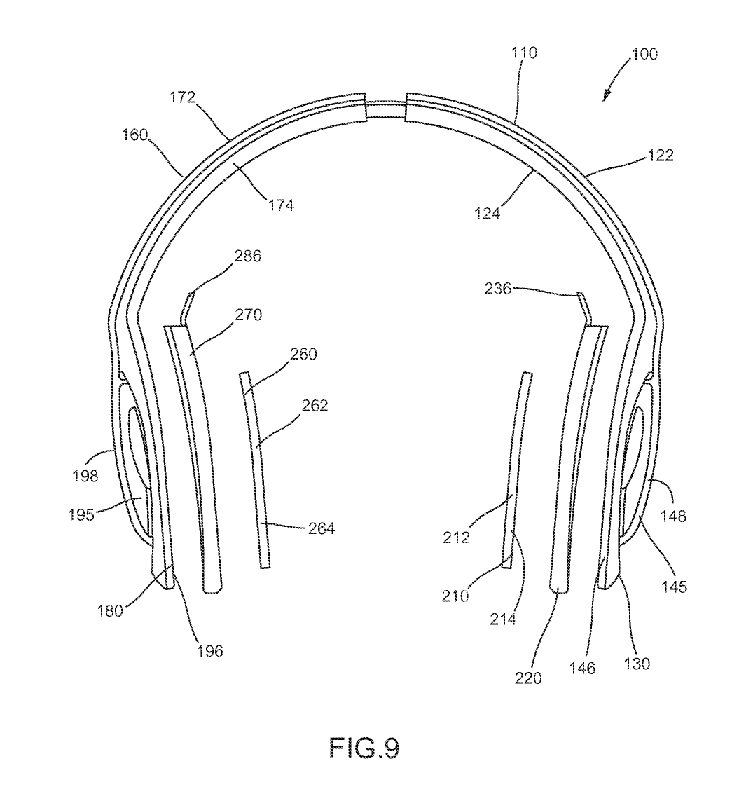

Referring to FIGS. 5-9, another embodiment of an ear warmer according to the present invention is illustrated. The ear warmer 100 is illustrated in a rear perspective view in FIG. 5 and in a top view in FIG. 6. The ear warmer 100 is also illustrated in an exploded configuration in the rear perspective view in FIG. 7, the front perspective view in FIG. 8, and the top perspective view in FIG. 9.

Referring to FIG. 5, the ear warmer 100 includes a frame that is configured to be worn by a user on the user's head. In this embodiment, the frame is adjustable in length so that the ear warmer 100 can be reconfigured for use by different users.

The frame has a frame portion 110 with an ear portion 130 proximate to an end of the frame portion 110. The frame portion 110 has a fabric portion 200 that is removably coupled thereto. As described in greater detail below, the fabric portion 200 includes a fabric member 210 that is coupled to a body 220. The body 220 is configured to be mounted or coupled onto the ear portion 130 and to be removed therefrom as desired, as described below.

The frame of the ear warmer 100 also has a frame portion 160 with an ear portion 180 proximate to an end of the frame portion 160. The frame portion 160 has a fabric portion 250 that is removably coupled thereto. The fabric portion 250 includes a fabric member 260 that is coupled to a body 270. The body 270 is configured to be mounted or coupled onto the ear portion 180 and to be removed therefrom as desired in a manner similar to body 220.

As illustrated in FIG. 5, the ear warmer 100 also includes a coupler 300 that is slideably received in an opening or slot formed in the end of each of the frame portion 110 and the frame portion 160. The mounting of the frame portions 110 and 160 to the coupler 300 allows for the manual adjustment of the frame portions 110 and 160 relative to each other to change the overall length of the frame of the ear warmer 100. In one implementation, the coupler 300 may include stops that limit the movement of the frame portions 110 and 160 to prevent the decoupling of the frame portions 110 and 160 from the coupler 300.

Referring to FIG. 6, frame portion 110 of ear warmer 100 has a proximal end 112 and an opposite distal end 114. Similarly, frame portion 160 has a proximal end 162 and an opposite distal end 164. Each of the proximal ends 112 and 162 is connected to the coupler 300, as described above. As illustrated, fabric portion 200 is mounted on the inner side of ear portion 130 so that fabric portion 200 engages and contacts the ear of the user. Similarly, fabric portion 250 is mounted on the inner side of ear portion 180 so that fabric portion 250 engages and contacts the ear of the user.

Referring to FIG. 7, the details of the fabric portions 200 and 250 of ear warmer 100 are illustrated. As mentioned above, fabric portion 200 includes a fabric member 210 and a body 220. The body 220 includes an inner edge 222 that defines an opening 224 extending through the body 220. The body 220 also includes a mounting structure 230 that has an edge or edge portion 232 defining a groove 234. The mounting structure 230 is made of a resilient material that can stretch as the mounting structure 230 is placed over the outer edge of the ear portion 130 and can return to its unbiased configuration with the outer edge of the ear portion 130 aligned with the groove 234. As a result, the fabric portion 200 is mounted to the ear portion 130 when the mounting structure 230 extends around a majority of or substantially all of the outer perimeter of the ear portion 130. To facilitate the decoupling of the fabric portion 200 from the ear portion 130, the body 220 includes a tab 236 that can be grasped and pulled by a user to decouple the mounting structure 230 from the ear portion 130.

Similarly, fabric portion 250 includes a fabric member 260 and a body 270. The body 270 includes an inner edge 272 that defines an opening 274 extending through the body 270. The body 270 also includes a mounting structure 280 that has an edge or edge portion 282 defining a groove 284. Like mounting structure 230, mounting structure 280 is made of a resilient material that can stretch as the mounting structure 280 is placed over the outer edge of the ear portion 180 with the outer edge of the ear portion 180 aligned with the groove 284. As a result, the fabric portion 250 is mounted to the ear portion 180 when the mounting structure 280 extends around a majority of or substantially all of the outer perimeter of the ear portion 180. Body 270 includes a tab 286 that can be grasped and pulled by a user to decouple the mounting structure 280 from the ear portion 180.

The end of band portion 160 includes a wall with an opening or slot 166 into which a portion of coupler 300 can be inserted as illustrated. The end of band portion 110 also includes a wall with an opening or slot similar to opening 166 into which a portion of coupler 300 can be inserted. Also shown in FIG. 7 is an upper surface 118 of the frame portion 110 and an opposite, lower surface 120 of the frame portion 110. Frame portion 160 also has an upper surface 168 and a lower surface 170.

Referring to FIG. 8, a front perspective view of the ear warmer 100 is illustrated in an exploded configuration. Ear portion 130 includes a perimeter member or frame 132 with an upper surface 134, a lower surface 136, an inner surface 138, and an outer surface 140. The frame 132 includes an inner edge 142 that defines an opening 144 extending through the frame 132. The frame 132 of the ear portion 130 includes a support member 145 that provides structural support to the frame 132.

Similarly, ear portion 180 includes a perimeter member or frame 182 with an upper surface 184, a lower surface 186, an inner surface 188, and an outer surface 190. The frame 182 includes an inner edge 192 that defines an opening 194 extending through the frame 182. The frame 182 of the ear portion 190 includes a support member 195 similar to support member 145.

The relative inner sides and outer sides of the various components of ear warmer 100 are illustrated in the exploded top view of FIG. 9. As shown, frame portion 110 has an outer side 122 and an inner side 124 and ear portion 130 has an inner side 146 and an outer side 148 with the support 145 located on the outer side 148. Similarly, frame portion 160 has an outer side 172 and an inner side 174 and ear portion 180 has an inner side 196 and an outer side 198 with the support 195 located on the outer side 198.

Each of the bodies 220 and 270 with tabs 236 and 286, respectively, are illustrated in FIG. 9. Fabric portion 210 has an inner side 212 and an outer side 214. Similarly, fabric portion 260 has an inner side 262 and an outer side 264. Fabric can be coupled via welding, an adhesive, co-molding, or other technique.

In this embodiment, each of the corresponding ones of the outer side or surface, the inner side or surface, the upper side or surface, and the lower side or surface of each of the frame portion 160 and the ear portion 180 forms a substantially continuous surface. In other words, the outer sides or surfaces of frame portion 160 and ear portion 180 form a substantially continuous surface, the inner sides or surfaces of frame portion 160 and ear portion 180 form a substantially continuous surface, the upper sides or surfaces of frame portion 160 and ear portion 180 form a substantially continuous surface, and the lower sides or surfaces of frame portion 160 and ear portion 180 form a substantially continuous surface. Similarly, each of the corresponding ones of the outer side or surface, the inner side or surface, the upper side or surface, and the lower side or surface of each of the frame portion 110 and the ear portion 130 forms a substantially continuous surface.

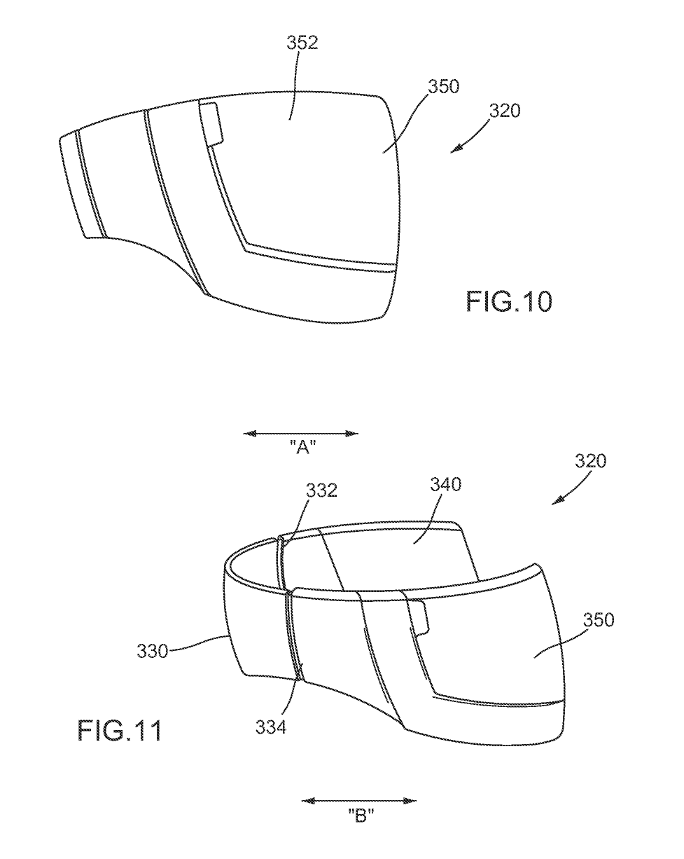

Referring to FIGS. 10 and 11, another embodiment of an ear warmer according to the present invention is illustrated. In this embodiment, the ear warmer 320 includes a band portion 330 with extensions 332 and 334 extending from opposite ends. The band portion 330 in ear warmer 320 can be formed of a rubber material or a rubber-coated material.

The ear warmer 320 includes an ear portion 340 that is slideably mounted onto extension 332 and movable along the directions of arrow “A.” The ear warmer 320 also includes an ear portion 350 that is slideably mounted onto extension 334 and movable along the directions of arrow “B.” Ear portion 350 can be formed of a plastic frame that is covered by a fabric material 352 such as that illustrated in FIG. 10. The ear portions 340 and 350 are independently slideable, which allows for the length of the frame to be adjusted. In addition, the locations of the adjustment points of the ear warmer 320 are on the sides of the ear warmer 320.

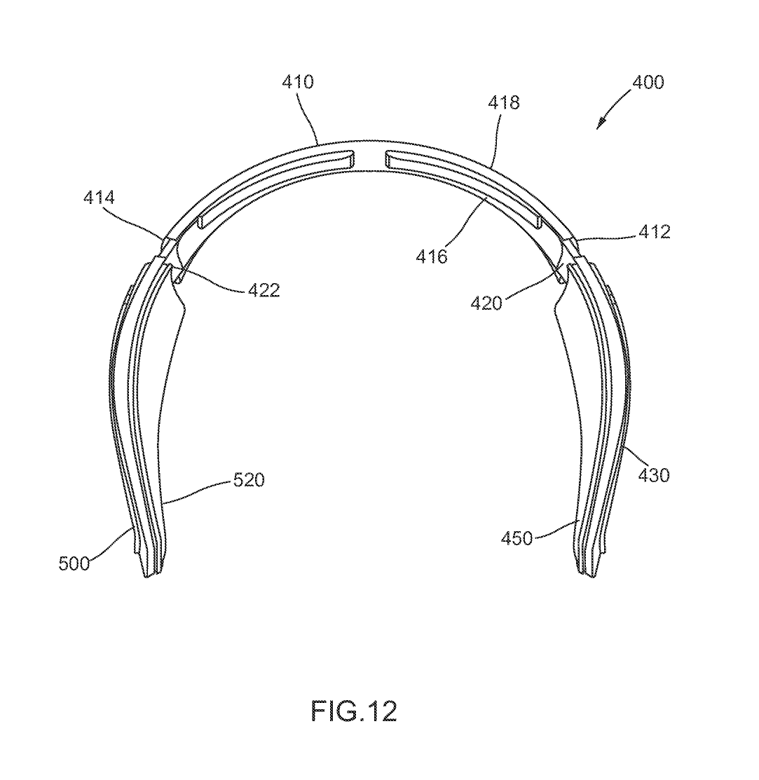

Another embodiment of an ear warmer according to the present invention is illustrated in FIGS. 12-15. The ear warmer 400 includes a band portion 410 and ear portions 430 and 500 that are slideably coupled to the band portion 410. The ear portions 430 and 500 are independently slideable on the band portion 410 and can be moved to adjust the overall length of the ear warmer 400.

The band portion 410 includes opposite ends 412 and 414 and an inner side 416 and an outer side 418. The band portion 410 also includes extensions 420 and 422 that extend from the ends 412 and 414, respectively. The ear portions 430 and 500 are slideably mounted on the extensions 420 and 422, respectively. As shown in FIG. 12, ear portion 430 includes a fabric portion 450 and ear portion 500 includes a fabric portion 520. The fabric portions 450 and 520 are positioned so that they contact the ears of the wearer of the ear warmer 400.

In an alternative embodiment, the ear portions 430 and 500 are pivotally coupled to the band portion 410, which enables the ear warmer 400 to be reconfigured into a collapsed configuration. In that embodiment, the extensions 420 and 422 can have hinges or joints that permit the ear portions 430 and 500 to pivot inwardly toward each other. As a result, ear portion 430 is pivotally coupled to part of the band portion 410 and ear portion 500 is pivotally coupled to part of the band portion 410. Referring to FIG. 14, ear portion 430 is pivotable about axis 421 and ear portion 500 is pivotable about axis 423.

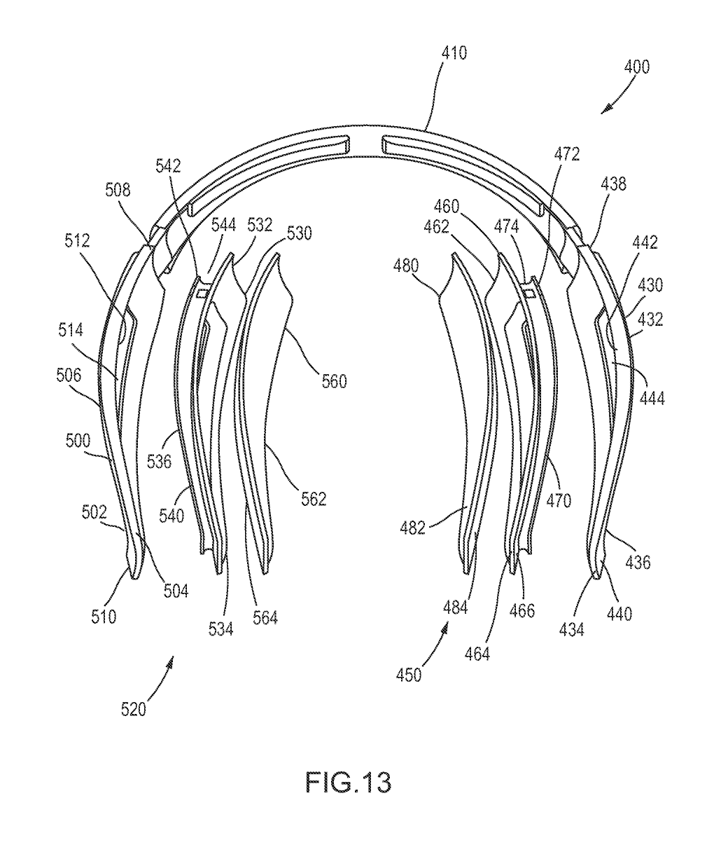

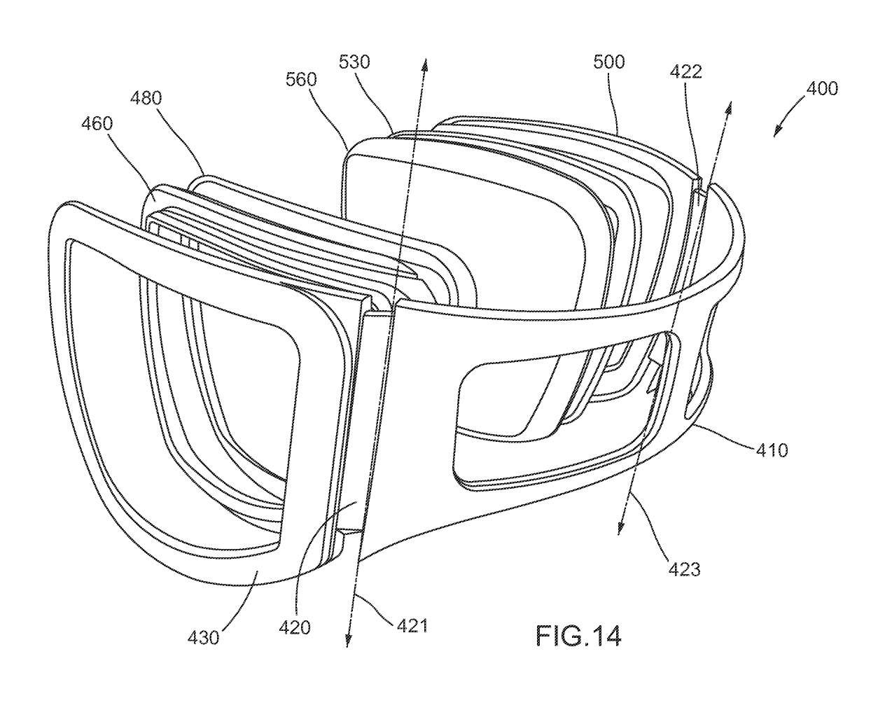

Referring to FIG. 13, the details of some of the components of the ear warmer 400 are illustrated. As shown, ear portion 430 includes a body or frame 432 that has an inner side 434, an outer side 436 opposite to the inner side 434, a proximal end 438 coupled to the band portion 410, and a distal end 440. The body 432 has an inner edge 442 that defines an opening 444 that extends through the body 432. Similarly, ear portion 500 includes a body or frame 502 that has an inner side 504, an outer side 506 opposite to the inner side 504, a proximal end 508 coupled to the band portion 410, and a distal end 510. The body 502 has an inner edge 512 that defines an opening 514 that extends through the body 502.

Also illustrated in FIG. 13 are the details of fabric portion 450 and fabric portion 520. Fabric portion 450 includes a mounting component 460 and fabric 480 that is fixedly coupled to the mounting component 460. The mounting component 460 includes a base 462 with an inner side 464 and an outer side 466. The mounting component 460 also includes a mounting portion 470 that is removably coupleable to the ear portion 430. In this embodiment, the mounting portion 470 includes a projection or mount 472 that defines a groove 474. The projection or mount 472 is sized so that is inserted into the opening 444 of the ear portion 430. The inner edge 442 of the ear portion 430 is inserted into the groove 474 of the mounting portion 470 and the lip of the mount 472 extends slightly onto the outer side 436 of the ear portion 430.

The fabric 480 can be co-molded with mounting component 460. Alternatively, fabric 480 can be welded to mounting component 460. Fabric 480 has an inner side 482 and an outer side 484. The fabric 480 can be one or more pieces of materials and can be formed of different materials. The layers of materials forming fabric 480 can vary and be located as desired based on the properties of the materials.

Similarly, fabric portion 520 includes a mounting component 530 and fabric 560 that is fixedly coupled to the mounting component 530. The mounting component 530 includes a base 532 with an inner side 534 and an outer side 536. The mounting component 530 also includes a mounting portion 540 that is removably coupleable to the ear portion 500. In this embodiment, the mounting portion 540 includes a projection or mount 542 that defines a groove 544. The projection or mount 542 is sized so that is inserted into the opening 514 of the ear portion 500. The inner edge 512 of the ear portion 500 is inserted into the groove 544 of the mounting portion 540 and the lip of the mounting portion 540 extends slightly onto the outer side 506 of the ear portion 500.

Like fabric 480, fabric 560 can be co-molded with mounting component 530. Alternatively, fabric 560 can be welded to mounting component 530. Fabric 560 has an inner side 562 and an outer side 564 and can be one or more layers.

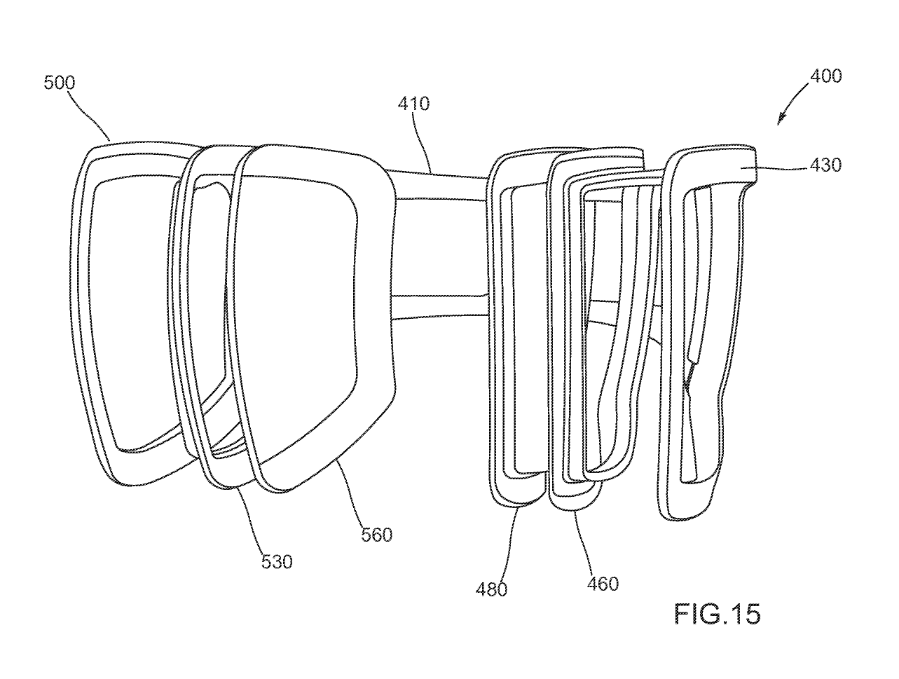

Referring to FIGS. 14 and 15, rear and front exploded perspective views are illustrated. As shown, the extensions 420 and 422 of band portion 410 of the ear warmer 400 have the ear portions 430 and 500 slideably coupled thereto. Mounting component 460 with fabric 480 is coupleable to ear portion 430 and mounting component 530 with fabric 560 is coupleable to ear portion 500.

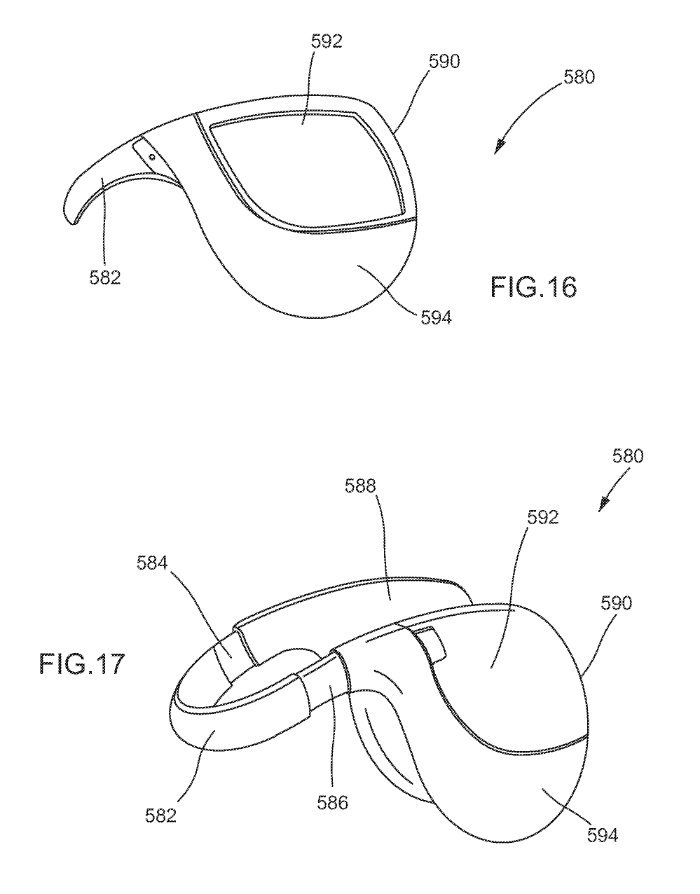

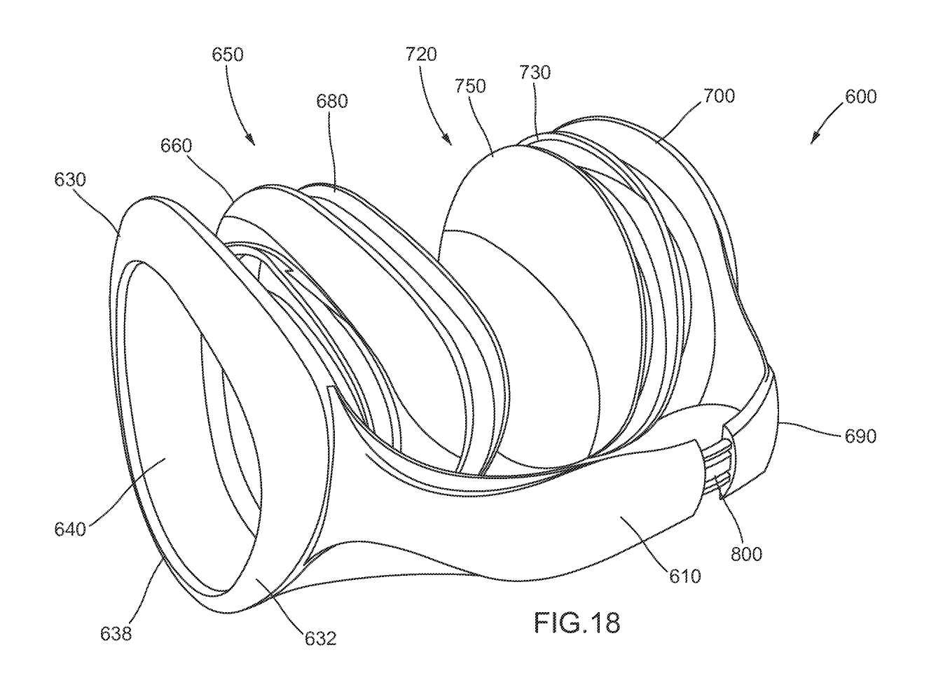

Referring to FIGS. 16 and 17, another embodiment of an ear warmer according to the present invention is illustrated. In this embodiment, the ear warmer 580 includes a band portion 582 with extensions 584 and 586 extending from opposite ends of the band portion 582. Ear portion 588 is slideably mounted onto extension 584. Similarly, ear portion 590 is slideably mounted onto extension 586. Ear portion 590 includes a molded portion 594 and a fabric portion 592 that collectively define the outer surface of the ear portion 590. Ear portion 588 has a similar combination of a molded portion and a fabric portion. In one implementation, the extensions 584 and 586 are molded into band portion 582. The extensions 584 and 586 can be plastic or metal or another material that is sufficiently strong to support the ear portions.

Another embodiment of an ear warmer according to the present invention is illustrated in FIGS. 18-20. The ear warmer 600 includes a band or frame portion 610 with an ear portion 630 proximate to one end. The ear warmer 600 also includes a band or frame portion 690 with an ear portion 700 proximate to one end. The band portions 610 and 690 are slideably mounted onto a coupler 800. The band portions 610 and 690 are independently adjustable relative to each other to change the overall length of the ear warmer 600.

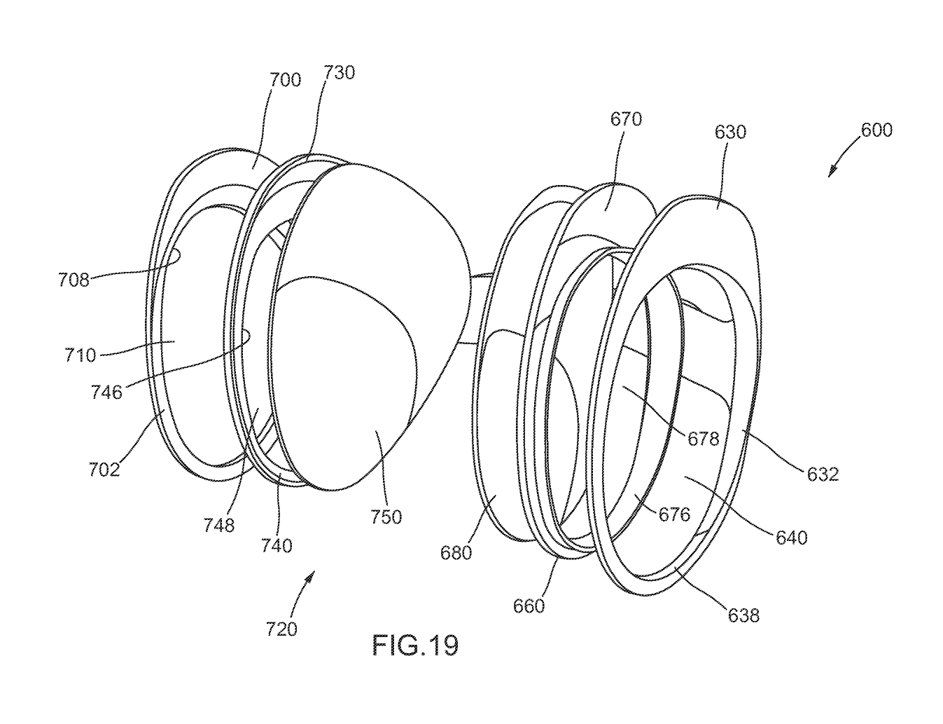

Referring to FIGS. 18 and 19, ear portion 630 includes a body 632 with an inner edge 638 that defines an opening 640 through the body 632. Ear portion 700 also includes a body 702 with an inner edge 708 that defines an opening 710 through the body 702. The ear warmer 600 includes a fabric portion 650 that has a mounting component 660 and fabric 680 coupled to the mounting component 660. The mounting component 660 includes a mounting portion 670 with an inner edge 676 that defines an opening 678. The ear warmer 600 also includes a fabric portion 720 that has a mounting component 730 and a fabric 750 coupled to the mounting component 730. The mounting component 730 includes a mounting portion 740 with an inner edge 746 that defines an opening 748.

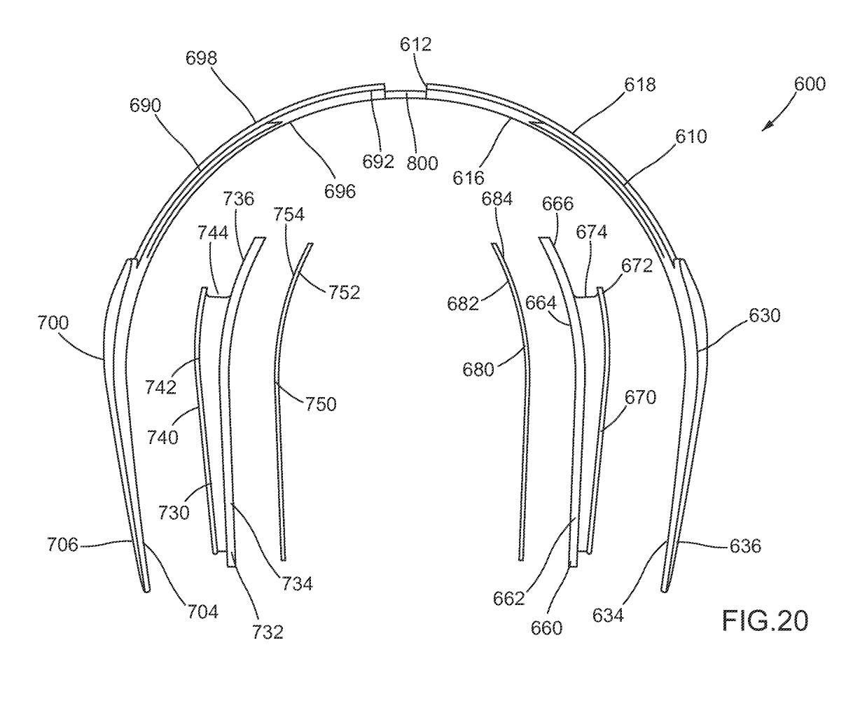

Referring to FIG. 20, an exploded view of the ear warmer 600 is illustrated. The band portion 610 and band portion 690 are slideably mounted to coupler 800 and adjustable to change the overall length of the ear warmer 600. Band portion 610 has an end 612, an inner side 616, and an outer side 618. The ear portion 630 of band portion 610 also has an inner side 634 and an outer side 636. Similarly, band portion 690 has an end 692, an inner side 696, and an outer side 698. The ear portion 700 of band portion 690 also has an inner side 704 and an outer side 706.

Fabric 680 has an inner side 684 and an outer side 686. Fabric 680 is fixedly coupled to the mounting component 660, such as via welding or co-molding or an adhesive. The mounting component 660 includes a base 662 with an inner side 664 and an outer side 666. The base 662 includes a mounting portion 670 extending therefrom that defines a groove 674 and a lip 672. The mounting portion 670 is sized so that it can be inserted into the opening 640 of the ear portion 630 with the inner edge 638 of the ear portion 630 being aligned with the groove 674 and the lip 672 extending slightly onto the outer side 636 of the body 632 of the ear portion 630.

Fabric 750 has an inner side 752 and an outer side 754. Fabric 750 is fixedly coupled to a mounting component 730 which includes a base 732 with an inner side 734 and an outer side 736. The base 732 includes a mounting portion 740 extending therefrom that defines a groove 744 and a lip 742. The mounting portion 740 is sized so that it can be inserted into the opening 710 of the ear portion 700 with the inner edge 708 of the ear portion 700 being aligned with the groove 744 and the lip 742 extending slightly onto the outer side 706 of the body 702 of the ear portion 700.

The disclosures of the following U.S. patents are hereby incorporated by reference in their entirety for all purposes: U.S. Pat. Nos. 5,835,609, 6,332,223, 6,499,146, 6,502,247, 6,502,248, 6,735,784, 7,212,645, 7,650,649, and 7,963,970.

While the invention has been described in detail and with references to specific embodiments thereof, it will be apparent to one skilled in the art that various changes and modifications can be made therein without departing from the spirit and scope thereof. Thus, it is intended that the present invention covers the modifications and variations of this invention.

What is claimed is:

1. An apparatus, comprising:
a first ear portion defining an opening, a wall of the first ear portion defining a groove therein;
a mounting component configured to engage the groove to removably couple the mounting component to the first ear portion;
a second ear portion;
a band disposed between the first ear portion and the second ear portion, the band including an adjustment mechanism configured to adjust a length of the band.

2. The apparatus of claim 1, further comprising:
a membrane coupled to the mounting component, the membrane configured to be disposed adjacent to an inner surface of the first ear portion when the mounting component is coupled to the first ear portion.

3. The apparatus of claim 1, further comprising a reflective material coupled to the mounting component.

4. The apparatus of claim 1, wherein
the band includes a first band end and a second band end, and
the adjustment mechanism has a first configuration and a second configuration, the adjustment mechanism configured to fixedly couple the first band end to the second band end when the adjustment mechanism is in its first configuration, the adjustment mechanism configured to slideably couple the first band end to the second band end when the adjustment mechanism is in its second configuration.

5. The apparatus of claim 1, further comprising an electronic communication device coupled to the band.

6. The apparatus of claim 1, further comprising:
an electronic communication device coupled to the band;
a speaker, electronically coupled to the electronic communication device, the speaker coupled to the second ear portion.

7. An apparatus comprising:
a first ear portion having an outer edge, the first ear portion defining an opening;
a mounting component having an edge defining a groove, the groove configured to engage the outer edge of the first ear portion, removably coupling the mounting component to the first ear portion;
a membrane coupled to the mounting component, the membrane configured to cover the opening when the mounting component is coupled to the first ear portion;
a second ear portion; and
a band coupled to and disposed between the first ear portion and the second ear portion.

8. The apparatus of claim 7, further comprising a coupler slideably received in an opening defined by an end of a first portion of the band, the coupler configured to couple the first portion of the band to a second portion of the band and including a stop configured to limit the movement of the first portion of the band relative to the second portion of the band.

9. The apparatus of claim 7, wherein the mounting component is constructed of resilient material configured to be stretched about the first ear portion.

10. The apparatus of claim 7, further comprising a tab coupled to the mounting component, the mounting component configured to be removed from the first ear portion when a force is applied to the tab.

11. The apparatus of claim 7, wherein the opening is a first opening,
the first ear portion includes a first extension,
the second ear portion includes a second extension, and
the band defines a second opening and a third opening, the first extension of the first ear portion configured to be slideably disposed within the second opening, the second extension of the second ear portion configured to be slideably disposed within the third opening.

12. The apparatus of claim 7, wherein the first ear portion and the second ear portion are pivotally coupled to the band.

13. The apparatus of claim 7, wherein the membrane and the mounting component are co-molded.

14. An apparatus comprising:
a first ear portion having an inner edge defining an opening;
a mounting component having an inner side and an outer side, a projection of the inner side and a lip of the outer side defining a groove, the inner edge of the first ear portion configured to be inserted into the groove removably coupling the mounting component to the first ear portion;
a membrane coupled to the inner side of the mounting component;
a second ear portion; and
a band portion, the band portion configured to be coupled to a proximal end of the first ear portion and a proximal end of the second ear portion.

15. The apparatus of claim 14, wherein the lip extends into the opening when the mounting component is coupled to the first ear portion.

16. The apparatus of claim 14, wherein the membrane is a fabric material configured to provide thermal insulation.

17. The apparatus of claim 14, wherein the second ear portion is pivotally coupled to the band.

18. The apparatus of claim 14, wherein the proximal end of the second ear portion is slideably coupled to the band.

19. The apparatus of claim 14, further comprising an adjustment mechanism, the adjustment mechanism having a first configuration and a second configuration, the adjustment mechanism configured to fixedly couple the proximal end of the second ear portion to the band when the adjustment mechanism is in its first configuration, the adjustment mechanism configured to slideably couple the second ear portion to the band when the adjustment mechanism is in its second configuration.

20. The apparatus of claim 14, further comprising an electronic communication device coupled to the band.
